(19) United States
(12) Patent Application Publication
(10) Pub. No.: US 2023/0137929 A1
(43) Pub. Date: May 4, 2023

(54) APPARATUS AND METHOD FOR MANUFACTURING MICRONEEDLE PATCH USING ELECTROHYDRODYNAMIC PRINTING

(71) Applicant: FEROKA INC., Seoul (KR)

(72) Inventors: In Duk LEE, Seoul (KR); Yeo Myung LIM, Goyang-si (KR); Yi Seul JEON, Seoul (KR); Hyung Joon RYU, Seoul (KR); Jae Joon LEE, Seoul (KR); Jin Geun PARK, Seoul (KR); Seo Won LEE, Seoul (KR)

(73) Assignee: FEROKA INC., Seoul (KR)

(21) Appl. No.: 18/088,848

(22) Filed: Dec. 27, 2022

Related U.S. Application Data
(63) Continuation of application No. PCT/KR2021/008202, filed on Jun. 29, 2021.

(30) Foreign Application Priority Data
Jun. 29, 2020 (KR) 10-2020-0078898

(51) Int. Cl. A61M 37/00

(52) U.S. Cl. CPC A61M 37/0015; A61M 2037/0053

(57) ABSTRACT

An apparatus and method for manufacturing microneedle using electrohydrodynamic printing are provided. The apparatus includes a substrate on which a printed microneedle is placed, a nozzle unit receiving a base material, which is a biocompatible material, as ink and discharging the ink to the substrate, a power unit supplying power to the nozzle unit, and a controller controlling the power unit so that the ink is dropped from the nozzle unit.

### CROSS-REFERENCE TO RELATED APPLICATIONS

This application is a continuation of International Application No. PCT/KR2021/008202 filed on Jun. 29, 2021, which claims to priority to Korean Patent Application No. 10-2020-0078898 filed on Jun. 29, 2020, the entire contents of which are herein incorporated by reference.

### TECHNICAL FIELD

The present disclosure relates to a microneedle patch and a microneedle patch manufacturing method using electrohydrodynamic printing.

### BACKGROUND ART

Drug injection into human body has traditionally been performed by needle injection, but needle injection causes great pain. Therefore, a non-invasive drug injection method has also been developed, but the amount of drug required is too high compared to the amount injected.

Due to this, a lot of research has been done on the drug delivery system (DDS), which has made further progress with the development of nanotechnology.

Unlike conventional needle injections, microneedles may penetrate skin without pain or trauma. In addition, since the microneedle must penetrate the stratum corneum of the skin, a certain degree of physical hardness may be required. In addition, an appropriate length may be required in order for bioactive substances to reach the epidermal layer or the dermal layer of the skin. In addition, in order for the bioactive substance of hundreds of microneedles to be effectively delivered into the skin, the skin permeability of the microneedle must be high and maintained for a certain period of time until dissolution after being inserted into the skin.

The microneedle may be made by injecting a material into a mold and drying it. However, it is difficult to manufacture a micro mold suitable for the size of the needle, and maintenance is difficult. A tensile method is a method of manufacturing microneedle by pulling the material of the microneedle and cutting off the middle, but this method causes pain when attached to the skin and has difficulty in forming a narrow arrangement of the microneedle. Studies to overcome the limitations of these conventional microneedle manufacturing methods are continuing.

### SUMMARY

### Technical Problem

The purpose of the present disclosure is to provide an apparatus and method for manufacturing microneedle patch that may elaborately and precisely manufacture microneedle patch with a high aspect ratio using electrohydrodynamic printing.

### Technical Solution to Problem

One aspect of the present disclosure provides a microneedle manufacturing apparatus using electrohydrodynamic printing, the microneedle manufacturing apparatus includes a substrate on which a printed microneedle is placed, a nozzle unit receiving a base material, which is a biocompatible material, as ink and discharging the ink to the substrate, a power unit supplying power to the nozzle unit, and a controller controlling the power unit so that the ink is dropped from the nozzle unit.

### Advantageous Effects of Disclosure

The microneedle patch manufacturing apparatus and microneedle patch manufacturing method according to the present disclosure may manufacture high-resolution microneedle patch using electrohydrodynamic (EHD) printing technology. Since biocompatible ink may be dropped onto a substrate finely and precisely, a microneedle with a sharp tip may be manufactured.

In the microneedle patch manufacturing apparatus and microneedle patch manufacturing method according to present disclosure, if the width or height of the microneedle falls within a preset range, the controller may manufacture needle tip very precisely by controlling the size or interval of the ink to be dropped.

The microneedle patch manufacturing apparatus and microneedle patch manufacturing method according to the present disclosure may manufacture a microneedle with a multi-layer structure by controlling an electric field, voltage or waveform according to the physical properties of the ink. In particular, the resolution of the microneedle may be increased by controlling the sharp tip of the microneedle very precisely.

### BRIEF DESCRIPTION OF DRAWINGS

FIG. 1 is a diagram illustrating a microneedle patch manufacturing apparatus according to an embodiment of the present disclosure.

FIG. 2 is a configuration diagram of FIG. 1.

FIG. 3 is a perspective view illustrating a microneedle patch manufactured by the manufacturing apparatus of FIG. 1.

FIG. 4 is a view illustrating a cross section of FIG. 3.

FIG. 5 is a cross-sectional view illustrating a part of FIG. 4.

FIGS. 6 and 7 are diagrams illustrating modified examples of FIG. 5.

FIG. 8 is a flowchart illustrating a microneedle patch manufacturing method according to another embodiment of the present disclosure.

FIG. 9 is a flowchart illustrating a microneedle patch manufacturing method according to another embodiment of the present disclosure.

### DETAILED DESCRIPTION

One aspect of the present disclosure provides a microneedle manufacturing apparatus using electrohydrodynamic printing, the microneedle manufacturing apparatus includes a substrate on which a printed microneedle is placed, a nozzle unit receiving a base material, which is a biocompatible material, as ink and discharging the ink to the substrate, a power unit supplying power to the nozzle unit, and a controller controlling the power unit so that the ink is dropped from the nozzle unit.

In addition, the microneedle manufacturing apparatus may further include a curing unit curing the microneedle placed on the substrate.

In addition, the nozzle unit sequentially discharges a first ink and a second ink, which are different base materials, to the substrate, and the microneedle may be formed in a multilayer structure on the substrate.

In addition, the controller may control voltage and waveform of the power unit depending on the physical properties of the first ink and the second ink.

In addition, an electric field is formed between the nozzle unit and the substrate, and an electric field formed when the first ink is dropped from the nozzle unit and an electric field formed when the second ink is dropped from the nozzle unit may be set differently.

In addition, the microneedle manufacturing apparatus may further include an image acquisition unit photographing the ink dropped from the nozzle unit.

Another aspect of the present disclosure may provide a microneedle manufacturing method using electrohydrodynamic printing, the microneedle manufacturing method includes forming an electric field between a nozzle unit and a substrate, supplying ink, which is a biocompatible material, to the nozzle unit, and

forming a microneedle in a height direction of the substrate by dropping the ink from the nozzle unit onto the substrate.

In addition, in forming the microneedle in the height direction of the substrate, a controller adjusts position of the substrate or the nozzle unit or an electric field between the nozzle unit and the substrate, so that a sharp tip of the microneedle may be placed the farthest away from a surface of the substrate.

In addition, the microneedle has a first needle portion and a second needle portion which are formed of different base materials, and in forming the microneedle in the height direction of the substrate, a first ink is dropped on the substrate to form the first needle portion, and then a second ink may be dropped on the first needle portion to form the second needle portion.

In addition, in forming the microneedle in the height direction of the substrate, the controller may set an electric field formed when the first ink is dropped from the nozzle unit and an electric field formed when the second ink is dropped from the nozzle unit to be different from each other.

Other aspects, features and advantages other than those described above will become apparent from the following detailed description of the drawings, claims and disclosure.

### BEST MODE

Hereinafter, the configuration and operation of present disclosure will be described in detail with reference to embodiments of present disclosure illustrated in the accompanying drawings.

Since the present disclosure may apply various transformations and may have various embodiments, specific embodiments are illustrated in the drawings and described in detail in the detailed description. Effects and characteristics of the present disclosure, and methods for achieving them will become clear with reference to the embodiments described later in detail together with the drawings. However, the present disclosure is not limited to the embodiments disclosed below and may be implemented in various forms.

Hereinafter, embodiments of the present disclosure will be described in detail with reference to the accompanying drawings, and when describing with reference to the drawings, the same or corresponding components are given the same reference numerals, and overlapping descriptions thereof will be omitted.

In the following embodiments, terms such as first and second are used for the purpose of distinguishing one component from another component without limiting meaning.

In the following examples, expressions in the singular number include plural expressions unless the context clearly dictates otherwise.

In the following embodiments, terms such as include or have mean that features or elements described in the specification exist, and do not preclude the possibility that one or more other features or elements may be added.

In the drawings, the size of components may be exaggerated or reduced for convenience of explanation. For example, since the size and thickness of each component shown in the drawings are arbitrarily shown for convenience of explanation, the present disclosure is not necessarily limited to the illustrated ones.

When an embodiment is otherwise implementable, a specific process sequence may be performed differently from the described sequence. For example, two processes described in succession may be performed substantially simultaneously, or may be performed in an order reverse to the order described.

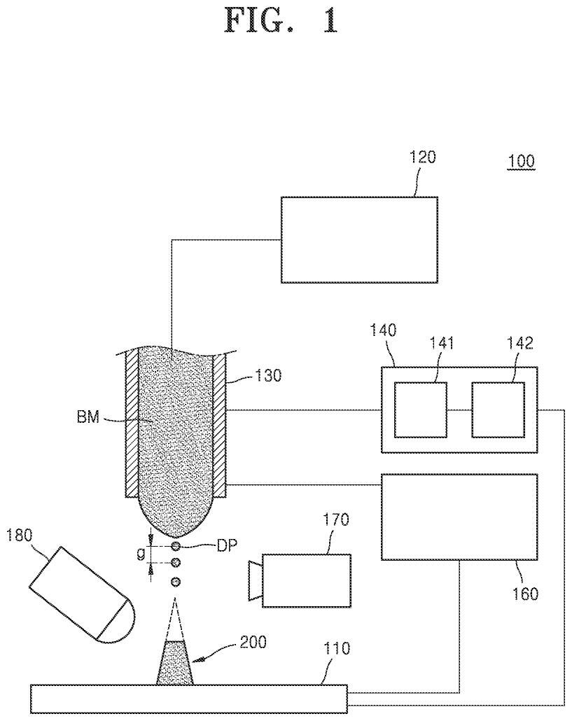

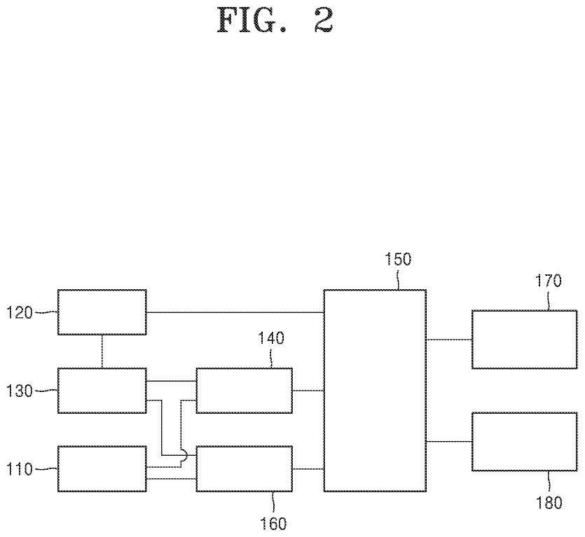

FIG. 1 is a diagram illustrating a microneedle patch manufacturing apparatus according to an embodiment of the present disclosure, and FIG. 2 is a configuration diagram of FIG. 1.

Referring to FIGS. 1 and 2, a microneedle patch manufacturing apparatus 100 is equipped with electrohydrodynamic (EHD) printing technology and may manufacture a microneedle patch with very high-resolution. The microneedle patch manufacturing apparatus 100 according to the present disclosure may precisely manufacture a microneedle and a microneedle patch using electrohydrodynamic jet 3D printing technology.

The microneedle patch manufacturing apparatus 100 may print both a base 210 and a microneedle 220 of a microneedle patch 200 described below. In addition, the microneedle patch manufacturing apparatus 100 may print the microneedle 220 by disposing the base 210 on a substrate 110 and dropping ink on the base 210.

The microneedle patch manufacturing apparatus 100 may include the substrate 110, a pump unit 120, a nozzle unit 130, a power unit 140, a controller 150, a positioning unit 160, an image acquisition unit 170 and a curing unit 180.

The substrate 110 is disposed under the nozzle unit 130, and the microneedle patch 200 printed with ink droplets DP ejected from the nozzle unit 130 may be placed. The printed microneedle may be placed on top of the substrate 110.

In one embodiment, the substrate 110 may be formed of a material at least partially conductive. The substrate 110 is electrically connected to the power unit 140, so that an electric field may be formed on top of the substrate 110.

In one embodiment, the position of the substrate 110 may be adjusted in a three-dimensional space. The substrate 110 is connected to the positioning unit 160 so that it may be positioned in space along 3 axes or rotated around 3 axes.

The pump unit 120 may store the ink and supply the ink to the nozzle unit 130. The pump unit 120 stores a base material BM, that is, a biocompatible material, which is a material of microneedle, as the ink. The pump unit 120 may be connected to the nozzle unit 130 to supply the base material BM to the nozzle unit 130.

In one embodiment, the pump unit 120 may include a syringe and a syringe pump. The base material is stored inside the syringe, and the syringe pump is driven so that the base material stored in the syringe may be supplied to the nozzle unit 130. The form of the pump unit 120 is not limited thereto, and may be set in various forms capable of storing and supplying the ink.

The nozzle unit 130 may receive the base material BM, which is the biocompatible material, as the ink and discharge the ink to the substrate 110. The nozzle unit 130 discharges the ink droplets DP from a nozzle head to the substrate 110, enabling print precise and high-resolution microneedle.

The nozzle unit 130 is connected to the power unit 140, and the electric field may be formed in a space between the nozzle unit 130 and the substrate 110. When an amplified voltage is supplied to the nozzle unit 130, the micron-sized ink droplets DP are discharged to the substrate 110.

In detail, the ink, which is the base material having electrical conductivity, is deposited on the nozzle head of the nozzle unit 130. When a high voltage of a voltage amplifier 141 is applied to the nozzle unit 130, the electric field is formed between the nozzle head and the substrate, and gravity and electric force act on the ink as external forces. When the sum of the external forces is greater than the surface tension of the ink, the ink is discharged as droplet.

The nozzle unit 130 may discharge multiple types of ink to the substrate 110. If the microneedle has a plurality of needle portions, the nozzle unit 130 may sequentially discharge a first ink and a second ink, which are different base materials, to the substrate 110 in order to print the plurality of needle portions. Thus, the microneedle may be formed as a multilayer structure on the substrate.

The nozzle unit 130 has a plurality of nozzles and may discharge the multiple types of ink or the same type of ink, so that a microneedle array of multilayer structure may be formed on the substrate. In one embodiment, two nozzles may form a first needle portion with the first ink, and the other two nozzles may form a second needle portion with the second ink.

The power unit 140 may supply power to the nozzle unit 130. The power unit 140 may be connected to the nozzle unit 130 and the substrate 110 to form the electric field between the substrate 110 and the nozzle unit 130.

The power unit 140 may include the voltage amplifier 141. The voltage amplifier 141 amplifies the supplied voltage, so that a large electric field may be formed between the nozzle unit 130 and the substrate 110.

The power unit 140 may include a waveform generator 142. The waveform generator 142 may control a waveform of current supplied to the nozzle unit 130 and the substrate 110, and the controlled waveform may adjust the interval g of the ink. The waveform generator 142 may adjust the interval g of the ink droplets DP by generating a waveform such as a square wave and adjusting the timing.

The controller 150 may be connected to the pump unit 120 to control driving of the pump unit 120. According to the control signal of the controller 150, the pump unit 120 may control the flow rate of the base material in the form of the ink supplied to the nozzle unit 130.

The controller 150 may control the power unit 140 to drop the ink from the nozzle unit 130. The controller 150 may control the voltage amplifier 141 of the power unit 140 to adjust the amount of voltage supplied to the nozzle unit 130 or the substrate 110. In addition, the controller 150 may control the waveform generator 142 of the power unit 140 to control the waveform of current or voltage supplied to the nozzle unit 130 or the substrate 110.

The controller 150 may control the power unit 140 to control the size of the ink droplets DP or the distance between the ink droplets DP. The controller 150 controls the power unit 140 to precisely and finely control the ink droplets DP discharged from the nozzle unit 130.

In one embodiment, the controller 150 may control the power unit 140 according to physical properties of the base material. For example, the base material used to manufacture the microneedle has biocompatibility, and has different viscosities or surface tensions. Accordingly, the controller 150 may control the amplified voltage of the voltage amplifier 141 according to the type of the base material BM supplied to the nozzle unit 130 to adjust the size of the electric field. In addition, the controller 150 may control the waveform of the waveform generator 142 according to the type of the base material BM supplied to the nozzle unit 130 to adjust the distance between ink droplets DP or the size of the ink.

In one embodiment, the controller 150 may control the power unit 140 in consideration of physical properties of a plurality of types of base materials BM.

For example, if the microneedle has a first needle portion and a second needle portion of a laminated structure, the first needle portion is formed of the first ink, and the second needle portion is formed of the second ink, the controller 150 may control the voltage and waveform of the power unit 140 in consideration of the physical properties of the first ink and the second ink.

In addition, the controller 150 may control the power unit 140 so that an electric field formed when the first ink is dropped on the nozzle unit 130 is set differently from an electric field formed when the second ink is dropped on the nozzle unit.

In one embodiment, the controller 150 includes a memory, and information on the size and waveform of power supplied from the power unit 140 according to the base material BM may be stored in the memory. When the base material BM is injected into the pump unit 120 or the nozzle unit 130, the controller 150 may automatically set the power unit 140 according to the injected base material BM or according to a user's selection.

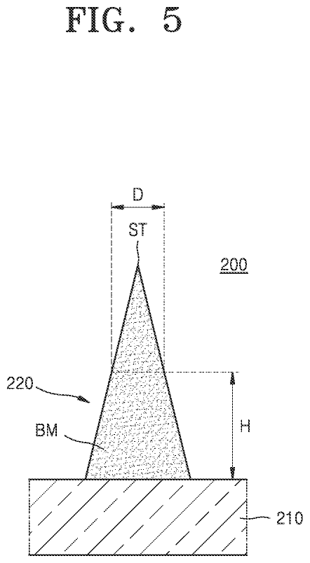

As an example, the controller 150 may control the power unit 140 according to the state of the printed microneedle. Referring to FIG. 5, the width D of the microneedle is formed to decrease as the height increases, and the ink must be precisely and elaborately dropped in the narrow portion of the width D. If the size of the ink is reduced or the interval g between the inks is increased, the microneedle may be printed precisely and elaborately.

When the microneedle reaches a preset height H or preset width D, the controller 150 controls the power unit 140 to precisely manufacture the end of the microneedle. The controller 150 may calculate the height H or width D of the microneedle to be printed based on an image captured by the image acquisition unit 170 or information on the amount of the ink discharged from the nozzle unit 130. If the calculated height H or width D corresponds to the preset range, the controller 150 may control the power unit 140 to adjust the size of the ink droplets DP or the distance between the ink droplets DP in order to precisely manufacture the microneedle.

The controller 150 controls the positioning unit 160 to adjust the spatial position of the substrate 110 or the height of the nozzle unit 130. By adjusting the spatial position of the substrate 110, the drop point of the ink dropped on the substrate 110 is changed, so that the microneedle may be precisely manufactured. In addition, by adjusting the height of the nozzle unit 130, the distance between the microneedle and the nozzle head may be controlled, so that the strength of the electric field or the falling speed of the ink may be controlled.

As an optional embodiment, the microneedle patch manufacturing apparatus 100 may include the image acquisition unit 170. The image acquisition unit 170 may be disposed adjacent to the substrate 110 to generate the image of the microneedle to be printed on the substrate 110. Also, the image acquisition unit 170 may capture the ink falling from the nozzle unit 130 to the substrate 110.

The controller 150 may receive image information generated by the image acquisition unit 170, and may extract information about ink size, ink interval, etc. from the transmitted image information. Also, the controller 150 may control the power unit 140 based on the extracted information.

In addition, the controller 150 may generate a signal initiating control of the power unit 140 based on the information about the width D or height H of the microneedle extracted from the image information. That is, when the controller 150 determines from the image information that the width D or height H of the microneedle falls within the preset range, the controller 150 controls the power unit 140 to control the ink size or interval.

As an alternative embodiment, the microneedle patch manufacturing apparatus 100 may include the curing unit 180. The curing unit 180 may cure the microneedle dropped or printed on the substrate 110.

For example, the curing unit 180 may include an optical module radiating light for curing the base material. As another example, the curing unit 180 may include a fan module to cure the base material, and may generate air flow by driving the fan module.

The controller 150 may quickly cure the microneedle placed on the substrate 110 by controlling the curing unit 180.

The microneedle patch manufacturing apparatus 100 according to the present disclosure may manufacture the high-resolution microneedle patch using electrohydrodynamic (EHD) printing technology. Since the biocompatible ink may be dropped onto the substrate finely and precisely, the microneedle with the sharp tip may be manufactured.

In the microneedle patch manufacturing apparatus 100 according to present disclosure, the controller 150 controls the power unit 140, so that the needle tip may be manufactured very precisely. If the width or height of the microneedle falls within the preset range, the controller 150 may manufacture the needle tip very precisely by controlling the size or interval of the ink to be dropped.

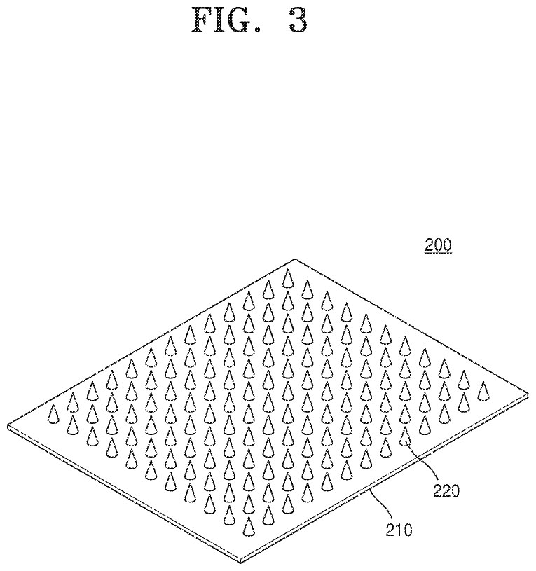

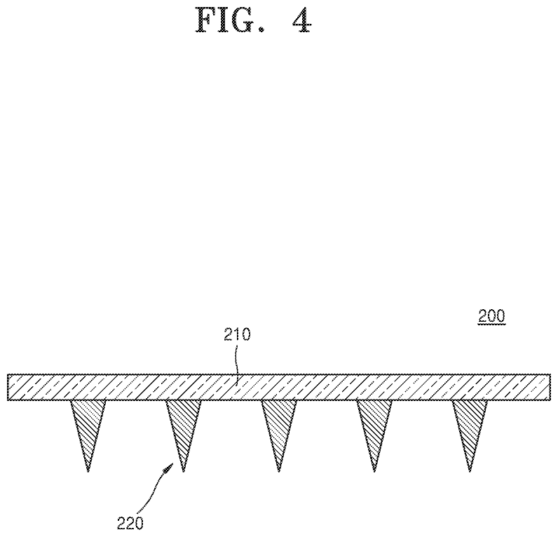

FIG. 3 is a perspective view illustrating a microneedle patch manufactured by the manufacturing apparatus of FIG. 1, FIG. 4 is a view illustrating a cross section of FIG. 3, and FIG. 5 is a cross-sectional view illustrating a part of FIG. 4.

Referring to FIGS. 3 to 5, in the microneedle patch 200 manufactured by the microneedle patch manufacturing apparatus 100, a plurality of microneedles 220 may be disposed on the base 210. The microneedle patch 200 may be attached to a subject to deliver drugs or cosmetic substances.

The base 210 supports the microneedle 220, and the plurality of microneedles 220 may be provided on one surface of the base. One side of the base 210 may be in contact with the skin, and the other side thereof may be exposed to the outside.

The base 210 may be removed once the microneedle 220 is implanted into the skin. For example, the base may be removed from the skin by applying force by the user. As another example, in the microneedle patch 200, a portion where the base 210 and the microneedle 220 are connected is first dissolved, and the base 210 may be removed after a certain time elapses after attachment. As another example, the base 210 of the microneedle patch 200 may dissolve when attached for a long time. As another example, the base 210 may be removed by applying a material for dissolution by the user.

In one embodiment, the base 210 may include any one of the materials included in the microneedle 220. The base 210 may include a biodegradable material like the microneedle 220.

As an alternative embodiment, the base 210 may include a bioactive substance. After the microneedle patch 200 is attached to the skin, active ingredients may be effectively delivered to a patient by the bioactive substance from the base 210. In addition, the base 210 and the microneedle 220 may be easily separated by the bioactive substance from the base 210.

In one embodiment, the base 210 may have a lower solubility than a layer closest to the base in the microneedle 220, that is, a layer most spaced apart from the tip of the microneedle 220. Since a portion closest to the base 210 in the microneedle 220 dissolves the fastest, the base 210 may be easily separated from the microneedle 220.

In one embodiment, the base 210 may include a water-soluble polymer. Base 210 may be composed of the water-soluble polymer or may contain other additives (e.g., disaccharides). In addition, the base 210 preferably does not contain drugs or active ingredients.

The base 210 may contain the biocompatible material. The biocompatible material selected as the base material of the microneedle 220 described later may be selected as the base material of the base 210.

The microneedle 220 protrudes from the surface of the base 210 and may be provided in plurality. The microneedle 220 is formed of the base material BM, and the base material BM may include the biocompatible material and additive.

The biocompatible material includes at least one of carboxymethyl cellulose (CMC), hyaluronic acid (HA), alginic acid, pectin, carrageenan, chondroitin sulfate, dextran sulfate, chitosan, polylysine, carboxymethyl chitin, fibrin, agarose, pullulan, polyanhydride, polyorthoester, polyetherester, polyesteramide, poly butyric acid, poly valeric acid, polyacrylate, ethylene-vinyl acetate polymer, acrylic substituted cellulose acetate, polyvinyl chloride, polyvinyl fluoride, polyvinyl imidazole, chlorosulphonate polyolefins, polyethylene oxide, polyvinylpyrolidone (PVP), hydroxypropyl methyl cellulose (HPMC), ethyl cellulose (EC), hydroxypropyl cellulose (HPC), carboxymethyl cellulose, cyclodextrin, maltose, lactose, trehalose, cellobiose, isomaltose, turanose and lactulose, or is at least one polymer selected from the group consisting of a copolymer of monomers forming such the polymer and cellulose.

The additive may include at least one of trehalose, oligosaccharide, sucrose, maltose, lactose, cellobiose, hyaluronic acid, alginic acid, pectin, carrageenan, chondroitin sulfate, dextran sulfate, chitosan, polylysine, collagen, gelatin, carboxymethyl chitin, fibrin, agarose, polyvinylpyrolidone (PVP), polyethylene glycol (PEG), polymethacrylate, hydroxypropyl methyl cellulose (HPMC), ethyl cellulose (EC), hydroxypropyl cellulose (HPC), carboxymethyl cellulose, cyclodextrin, gentiobiose, alkyltrimethylammonium bromide (cetrimide), hexadecyltrimethylammoniumbromide (CTAB), gentian violet, benzethonium chloride, docusate sodium salt, SPAN-type surfactant, polysorbate (Tween), sodium dodecyl sulfate (SDS), benzalkonium chloride and glyceryl oleate.

Hyaluronic acid is used as meaning including not only hyaluronic acid but also hyaluronic acid salts (e.g., sodium hyaluronate, potassium hyaluronate, magnesium hyaluronate and calcium hyaluronate) and mixtures thereof. Hyaluronic acid is used as meaning including cross-linked hyaluronic acid and/or non-cross-linked hyaluronic acid.

According to one embodiment of the present disclosure, the hyaluronic acid of the present disclosure has a molecular weight of 2 kDa to 5000 kDa.

According to another embodiment of the present disclosure, the hyaluronic acid of the present disclosure has a molecular weight of 100-4500 kDa, 150-3500 kDa, 200-2500 kDa, 220-1500 kDa, 240-1000 kDa or 240-490 kDa.

As carboxymethyl cellulose (CMC), CMC of various known molecular weights may be used. For example, the average molecular weight of CMC used in the present disclosure is 90,000 kDa, 250,000 kDa or 700,000 kDa.

The disaccharide may include sucrose, lactulose, lactose, maltose, trehalose, or cellobiose, and may include sucrose, maltose, or trehalose in particular.

As an optional embodiment, an adhesive may be included. Adhesive is one or more adhesives selected from a group consisting of silicone, polyurethane, hyaluronic acid, physical adhesive (gecko), polyacrylic, ethyl cellulose, hydroxymethyl cellulose, ethylene vinyl acetate and polyisobutylene.

As an alternative embodiment, the microneedle 220 may additionally include metal, high molecular weight polymer, or adhesive.

The microneedle 220 may have various shapes. The microneedle 220 may have a cone shape. For example, the microneedle 220 may have a polygonal shape such as a conical shape, a triangular pyramid shape, or a square pyramid shape. In addition, although the drawings illustrate that the microneedle 220 disposed on the microneedle patch 200 has the same shape, it is not limited thereto and may have different shapes.

The microneedle 220 has a very high aspect ratio at the tip. Therefore, the tip of the microneedle 220 must be manufactured very precisely. When the microneedle 220 reaches the preset height H or preset width D, the sharp tip may be formed by finely adjusting the size or interval of ink dropped into the microneedle 220.

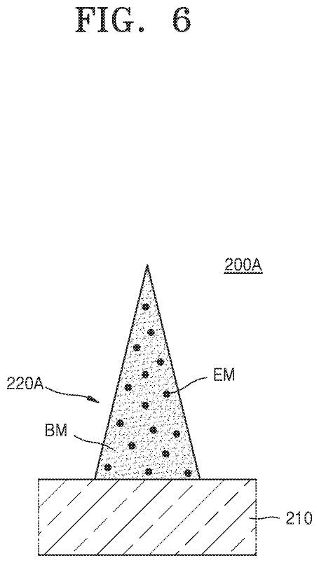

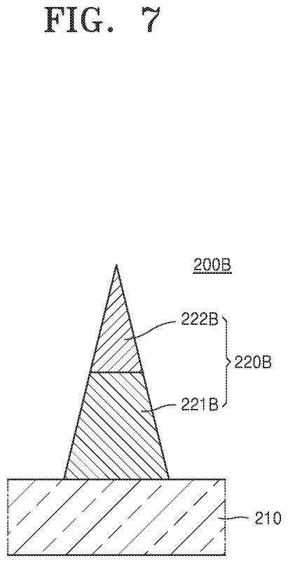

FIGS. 6 and 7 are diagrams illustrating modified examples of FIG. 5.

Referring to FIG. 6, a microneedle patch 200A includes a base 210 and a microneedle 220A, and the microneedle 220A may include an active ingredient EM.

The microneedle patch manufacturing apparatus 100 may print the microneedle 220A using base material BM mixed with the active ingredient EM as ink.

The microneedle 220A may contain, at least in part, the pharmaceutical, medical or cosmetic active ingredient EM. For example, as a non-limiting example, the active ingredient include protein/peptide drugs, but it is not limited thereto, and include at least one of hormones, hormone analogs, enzymes, enzyme inhibitors, and signal transduction proteins or portions thereof, antibodies or portions thereof, single-chain antibodies, binding proteins or binding domains thereof, antigens, adhesion proteins, structural proteins, regulatory proteins, toxin proteins, cytokines, transcriptional regulators, blood clotting factors and vaccines. More specifically, the protein/peptide drug may include any one of insulin, insulinlike growth factor 1 (IGF-1), growth hormone, erythropoietin, granulocyte-colony stimulating factors (G-CSFs), granulocyte/macrophage-colony stimulating factors (GM-CSFs), interferon alpha, interferon beta, interferon gamma, interleukin-1 alpha and beta, interleukin-3, interleukin-4, interleukin-6, interleukin-2, epidermal growth factors (EGFs), calcitonin, adrenocorticotropic hormone (ACTH), tumor necrosis factor (TNF), atobisban, buserelin, cetrorelix, deslorelin, desmopressin, dynorphin A (1-13), elcatonin, eleidosin, eptifibatide, growth hormone releasing hormone-II (GHRHII), gonadorelin, goserelin, histrelin, leuprorelin, lypressin, octreotide, oxytocin, pitressin, secretin, sincalide, terlipressin, thymopentin, thymosine, triptorelin, bivalirudin, carbetocin, cyclosporine, exedine, lanreotide, luteinizing hormone releasing hormone (LHRH), nafarelin, parathyroid hormone, pramlintide, enfuvirtide (T-20), thymalfasin and ziconotide. In addition, the active ingredient EM may be a cosmetic ingredient such as whitening, filler, wrinkle removal or antioxidant.

In one embodiment, the active ingredient EM may be a colloid dispersed in a solvent forming the microneedle 220A in the form of particulates. The particulate may itself be the active ingredient EM or may include a coating material carrying the active ingredient EM.

The active ingredient EM may be intensively distributed on some layers of the microneedle 220A. That is, since the active ingredient EM is placed at a specific height in the microneedle 220A, the active ingredient EM may be effectively delivered.

In another embodiment, the active ingredient EM may be dissolved in the microneedle 220A. The active ingredient EM may be dissolved in the base material of the microneedle 220A, such as the aforementioned biodegradable materials, to constitute the microneedle 220A. The active ingredient EM may be dissolved in the base material at an even concentration and may be intensively distributed at a specific height of the microneedle 220A like the particulate described above.

In one embodiment, the microneedle patch 200A may have a plurality of active ingredients EM according to zones. A microneedle of a first group among a plurality of microneedles may include a first active ingredient among the plurality of active ingredients, and a microneedle of a second group different from the first group may include a second active ingredient among the plurality of active ingredients.

In one embodiment, a pharmaceutical, medical or cosmetic active ingredient EM may be coated on the microneedle 220A. The active ingredients EM may be coated on the entire microneedle 220A or only a portion of the microneedle 220A. Alternatively, in the microneedle 220A, part of a coating layer may be coated with the first active ingredient, and the other part may be coated with the second active ingredient.

Referring to FIG. 7, a microneedle patch 200B may include a base 210 and a microneedle 220B.

FIG. 7 illustrates that a first needle portion 221B and a second needle portion 222B of the microneedle 220B have a layered structure, but it is not limited thereto and may have various shapes. For example, the first needle portion 221B and the second needle portion 222B may each have a different unique shape. However, in the following, for convenience of description, an embodiment in which the microneedle 220B has the layered structure will be mainly described.

The microneedle 220B may have a plurality of stacked layers. The number of layers constituting the microneedle 220B is not limited to a specific number, but hereinafter, for convenience of explanation, the microneedle 220B will be described based on an embodiment having the first needle portion 221B and the second needle portion 222B of a layered structure.

The first needle portion 221B and the second needle portion 222B may be formed of different base materials. The first needle portion 221B may be formed of a first ink as a first base material, and the second needle portion 222B may be formed of a second ink as a second base material different from the first base material.

The microneedle patch manufacturing apparatus 100 may print the first needle portion 221B by first dropping the first ink, and then may print the second needle portion 222B by dropping the second ink on the first needle portion 221B.

Since the physical properties of the first ink and the second ink are different, the controller 150 may control the power unit 140 according to each physical property.

In detail, in order to print the first needle portion 221B, the controller 150 may control the power unit 140 to set the voltage level and waveform according to the characteristics of the first ink. In addition, in order to print the second needle portion 222B, the controller 150 may control the power unit 140 to set the voltage level and waveform according to the characteristics of the second ink.

At this time, since the second needle portion 222B should be more pointed than the first needle portion 221B and have a smaller width D, the second ink should be precisely dropped from the nozzle unit 130. The controller 150 may control the voltage and/or waveform of the power unit 140 in consideration of the aspect ratio of the second needle portion 222B.

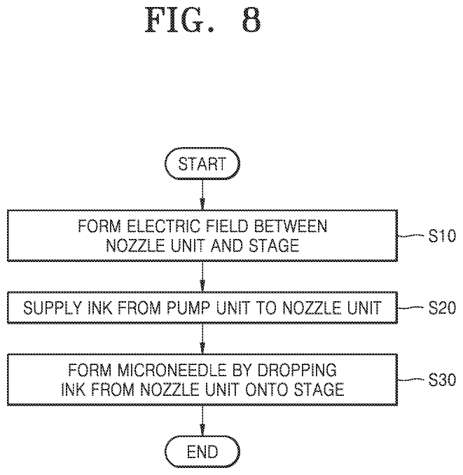

FIG. 8 is a flowchart illustrating a microneedle patch manufacturing method according to another embodiment of the present disclosure.

Referring to FIG. 8, a microneedle patch manufacturing method may include forming the electric field between the nozzle unit and the substrate (S10), supplying the ink, which is the biocompatible material, to the nozzle unit (S20), and forming the microneedle in the height direction of the substrate by dropping the ink from the nozzle unit onto the substrate (S30).

In forming the electric field between the nozzle unit and the substrate (S10), a strong electric field may be formed between the nozzle unit 130 and the substrate 110. The power unit 140 may apply a voltage to the substrate 110 and the nozzle unit 130 to form the strong electric field between the substrate 110 and the nozzle unit 130. The controller 150 may control the power unit 140 to control the size and waveform of power applied to the substrate 110 and the nozzle unit 130.

In supplying the ink, which is the biocompatible material, to the nozzle unit (S20), the ink is supplied to the nozzle unit 130. The base material used as the material of the microneedle is the biocompatible material, and is stored in the pump unit 120 as the ink. When a drive signal of the pump unit 120 is transmitted by the controller 150, the ink may be supplied from the pump unit 120 to the nozzle unit 130.

In forming the microneedle in the height direction of the substrate by dropping the ink from the nozzle unit onto the substrate (S30), the ink is dropped on the substrate 110 so that the microneedle may be printed. When the ink is continuously deposited on top of the substrate 110, the sharp tip of the microneedle may be placed at the most distant part from the surface of the substrate.

The controller 150 may control the positioning unit 160 to adjust the position of the substrate 110 or the nozzle unit 130. Also, the controller 150 may control the electric field between the nozzle unit 130 and the substrate 110. By the control signal of the controller 150, the microneedle may be manufactured very elaborately and precisely.

The microneedle patch manufacturing method according to the present disclosure may elaborately and precisely manufacture the microneedle patch with a high aspect ratio using electrohydrodynamic printing.

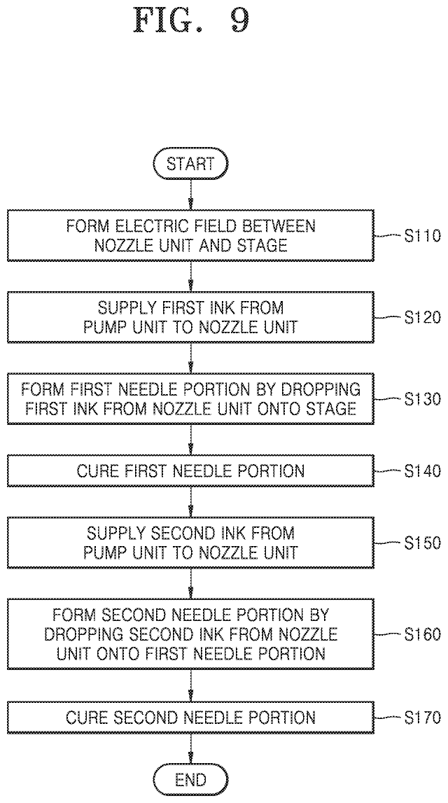

FIG. 9 is a flowchart illustrating a microneedle patch manufacturing method according to another embodiment of the present disclosure.

Referring to FIG. 9, the microneedle patch manufacturing method may include forming the electric field between the nozzle unit and the substrate (S110), supplying the first ink from the pump unit to the nozzle unit (S120), forming the first needle portion by dropping the first ink from the nozzle unit onto the substrate (S130), curing the first needle portion (S140), supplying the second ink from the pump unit to the nozzle unit (S150), forming the second needle portion by dropping the second ink from the nozzle unit onto the first needle portion (S160), and curing the second needle portion (S170).

The microneedle patch manufactured by the manufacturing method may have the layered structure formed of the plurality of base materials. That is, the microneedle may include the first needle portion formed of the first ink and the second needle portion formed of the second ink. Accordingly, the manufacturing method may form the first needle portion by dropping the first ink onto the substrate, and then form the second needle portion by dropping the second ink onto the first needle portion.

In forming the electric field between the nozzle unit and the substrate (S110), a strong electric field may be formed between the nozzle unit 130 and the substrate 110 by driving the power unit 140.

In supplying the first ink from the pump unit to the nozzle unit (S120), the first ink is supplied to the nozzle unit 130 to form the first needle portion 221B.

In forming the first needle portion by dropping the first ink from the nozzle unit onto the substrate (S130), the first needle portion 221B may be formed by dropping the first ink. At this time, the controller 150 may control the electric field according to the physical properties of the first ink. The controller 150 may control the voltage and waveform by controlling the power unit 140 according to the physical properties of the first ink. Also, the controller 150 may control the size and interval of the first ink droplets in consideration of the height and width of the first needle portion 221B or the aspect ratio of the first needle portion 221B.

In curing the first needle portion (S140), the first needle portion 221B may be cured using the curing unit 180. The curing unit 180 may irradiate light or drive the fan in consideration of the curing characteristics of the first ink.

In supplying the second ink from the pump unit to the nozzle unit (S150), the second ink is supplied to the nozzle unit 130 to form the second needle portion 222B.

In forming the first needle portion by dropping the second ink from the nozzle unit onto the first needle portion (S160), the second needle portion 222B may be formed by dropping the second ink. At this time, the controller 150 may control the electric field according to the physical properties of the second ink. The controller 150 may control the voltage and waveform by controlling the power unit 140 according to the physical properties of the second ink. In addition, the controller 150 may control the size and interval of second ink droplets in consideration of the height and width of the second needle portion 222B or the aspect ratio of the second needle portion 222B.

The controller 150 may set the electric field formed when the first ink is dropped from the nozzle unit 130 and the electric field formed when the second ink is dropped from the nozzle unit 130 differently.

In curing the second needle portion (S170), the second needle portion 222B may be cured using the curing unit 180. The curing unit 180 may irradiate light or drive the fan in consideration of curing characteristics of the second ink.

The microneedle patch manufacturing method according to the present disclosure may elaborately and precisely manufacture the microneedle patch with a high aspect ratio using electrohydrodynamic printing.

As such, present disclosure has been described with reference to the embodiments shown in the drawings, but this is only exemplary, and those skilled in the art will understand that various modifications and equivalent other embodiments are possible therefrom. Therefore, the true technical protection scope of the present disclosure should be determined by the technical spirit of the appended claims.

Specific executions described in the embodiments are examples, and do not limit the scope of the embodiments in any way. In addition, if there is no specific mention, such as “essential” or “important”, it may not be a necessary component for the application of present disclosure.

In the specification of the embodiments (particularly in the claims), the use of the term “above” and similar indicating terms may correspond to both singular and plural. In addition, when a range is described in an embodiment, it includes disclosure applying individual values belonging to the range (unless there is a description to the contrary), and it is the same as describing each individual value constituting the range in the detailed description. Finally, if there is no explicit description or description of the order of steps constituting the method according to the embodiment, the steps may be performed in an appropriate order. Examples are not necessarily limited according to the order of description of the steps. The use of all examples or exemplary terms (e.g., etc.) in the embodiments is simply for explaining the embodiments in detail, and the scope of the embodiments is limited due to the examples or exemplary terms unless limited by the claims. It is not. In addition, those skilled in the art can appreciate that various modifications, combinations and changes may be made according to design conditions and factors within the scope of the appended claims or equivalents thereof.

### INDUSTRIAL APPLICABILITY

According to an embodiment of the present disclosure, an apparatus and method for manufacturing microneedle patch using electrohydrodynamic printing may be applied to an apparatus and method for manufacturing microneedle.

What is claimed is:

1. A microneedle manufacturing apparatus using electrohydrodynamic printing, the microneedle manufacturing apparatus comprising:
a substrate on which a printed microneedle is placed;
a nozzle unit receiving a base material, which is a biocompatible material, as ink and discharging the ink to the substrate;
a power unit supplying power to the nozzle unit; and
a controller controlling the power unit so that the ink is dropped from the nozzle unit.

2. The microneedle manufacturing apparatus of claim 1, further comprising
a curing unit curing the microneedle placed on the substrate.

3. The microneedle manufacturing apparatus of claim 1, wherein
the nozzle unit sequentially discharges a first ink and a second ink, which are different base materials, to the substrate, and
the microneedle is formed in a multilayer structure on the substrate.

4. The microneedle manufacturing apparatus of claim 3, wherein
the controller controls voltage and waveform of the power unit depending on physical properties of the first ink and the second ink.

5. The microneedle manufacturing apparatus of claim 3, wherein
an electric field is formed between the nozzle unit and the substrate, and
an electric field formed when the first ink is dropped from the nozzle unit and an electric field formed when the second ink is dropped from the nozzle unit are set differently.

6. The microneedle manufacturing apparatus of claim 1, further comprising
an image acquisition unit photographing the ink dropped from the nozzle unit.

7. A microneedle manufacturing method using electrohydrodynamic printing, the microneedle manufacturing method comprising:
forming an electric field between a nozzle unit and a substrate;
supplying ink, which is a biocompatible material, to the nozzle unit; and
forming a microneedle in a height direction of the substrate by dropping the ink from the nozzle unit onto the substrate.

8. The microneedle manufacturing method of claim 7, wherein in forming the microneedle in the height direction of the substrate,
a controller adjusts a position of the substrate or the nozzle unit or an electric field between the nozzle unit and the substrate, so that a sharp tip of the microneedle is placed the farthest away from a surface of the substrate.

9. The microneedle manufacturing method of claim 7, wherein
the microneedle has a first needle portion and a second needle portion which are formed of different base materials, and
in forming the microneedle in the height direction of the substrate,
a first ink is dropped on the substrate to form the first needle portion, and then a second ink is dropped on the first needle portion to form the second needle portion.

10. The microneedle manufacturing method of claim 9, wherein
in forming the microneedle in the height direction of the substrate,
a controller sets an electric field formed when the first ink is dropped from the nozzle unit and an electric field formed when the second ink is dropped from the nozzle unit to be different from each other.
